(19) United States
(12) Patent Application Publication
(10) Pub. No.: US 2023/0218886 A1
(43) Pub. Date: Jul. 13, 2023

(54) SHEATHING AND DELIVERY SYSTEM FOR COLLAPSIBLE BLOOD PUMPS

(71) Applicant: Shifamed Holdings, LLC, Campbell, CA (US)

(72) Inventors: Janine ROBINSON, Half Moon Bay, CA (US); Ari RYAN, Sunnyvale, CA (US); Daniel VARGHAI, Campbell, CA (US); Crissly CRISOSTOMO, Campbell, CA (US); Ha LUONG, Campbell, CA (US); Jonelle BALDUCCI, Campbell, CA (US)

(21) Appl. No.: 18/152,551

(22) Filed: Jan. 10, 2023

Related U.S. Application Data
(60) Provisional application No. 63/297,972, filed on Jan. 10, 2022.
(60) Provisional application No. 63/267,467, filed on Feb. 2, 2022.
(60) Provisional application No. 63/376,375, filed on Sep. 20, 2022.

(51) Int. Cl. A61M 60/408; A61M 60/126

(52) U.S. Cl. CPC A61M 60/408; A61M 60/126; A61M 60/808

(57) ABSTRACT

A system for inserting a collapsible blood pump into a patient. In some embodiments, the system includes an introducer, the introducer comprising an introducer hub and an introducer sheath extending distally from the introducer hub, the introducer sheath comprising an introducer sheath lumen, the introducer hub comprising a hub connector and a distal hub lumen surrounding a proximal end of the introducer shaft; and a transfer tool comprising a transfer sheath, the transfer sheath comprising a transfer sheath lumen having a diameter substantially equal to a diameter of the introducer sheath lumen and a transfer tool connector adapted to connect to the hub connector, a distal portion of the transfer sheath extending into the distal hub lumen when the transfer tool connector is connected to the hub connector.

### CROSS REFERENCE TO RELATED APPLICATIONS

This application claims the benefit of U.S. Application No. 63/297,972, filed Jan. 10, 2022; U.S. Application No. 63/267,467, filed Feb. 2, 2022; and U.S. Application No. 63/376,375, filed Sep. 20, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Intravascular blood pumps may benefit from being collapsible to facilitate a smaller delivery profile prior to being expanded at the pumping site within the patient's heart and/or vasculature. Some such pumps are described in WO 2021/243263.

Conventional expandable blood pump systems require both an outer sheath and an introducer. The outer sheath facilitates collapse of the self-expanding pump and expansion to the operational profile. The pump is introduced into a patient by collapsing the pump into the sheath and then inserting the sheath through an introducer. This leads to “stacking” of materials and thus a larger insertion profile, which correlates to higher vascular complication rates. There is a need for a blood pump and delivery system having a lower insertion profile and larger operational configuration. There is a need for a system for quickly introducing an expandable blood pump into a patient.

### SUMMARY OF THE DISCLOSURE

The disclosure is related to intravascular blood pumps and methods of their use. In particular, the disclosure is related to systems and methods for inserting a collapsible blood pump into a patient.

One aspect of the invention provides a system for inserting a collapsible blood pump into a patient. In some embodiments, the system includes an introducer, the introducer having an introducer hub and an introducer sheath extending distally from the introducer hub, the introducer sheath having an introducer sheath lumen, the introducer hub having a hub connector and a distal hub lumen surrounding a proximal end of the introducer shaft; and a transfer tool having a transfer sheath, the transfer sheath having a transfer sheath lumen with a diameter substantially equal to a diameter of the introducer sheath lumen and a transfer tool connector adapted to connect to the hub connector, a distal portion of the transfer sheath extending into the hub when the transfer tool connector is connected to the hub connector.

In some embodiments, the distal portion of the transfer sheath extends into the distal hub lumen when the transfer tool connector is connected to the hub connector. In some embodiments, the distal end of the transfer sheath abuts a proximal end of the introducer sheath when the transfer tool connector is connected to the hub connector. In some embodiments, the introducer hub also has a tapered surface extending proximally and radially outwardly from the distal hub lumen.

In some embodiments, the introducer also has a one-way valve disposed in the introducer hub proximal to the introducer sheath lumen and configured to seal against vascular pressure, a seal disposed in the introducer hub proximal to the introducer sheath and configured to seal against vascular pressure around a range of diameters of devices inserted through the seal, and/or a disc valve disposed in the introducer hub proximal to the introducer sheath and configured to seal against vascular pressure around a range of diameters of devices inserted through the valve. In some embodiments, the introducer hub also has a purge fluid port in fluid communication with the distal hub lumen.

In some embodiments, the hub connector includes threads disposed on the introducer hub. In some embodiments, the hub connector and transfer tool connector are configured to provide an axial force to move the transfer sheath and introducer sheath toward each other.

In some embodiments, the transfer tool also has a proximal hub surrounding a proximal portion of the transfer sheath. In some such embodiments, the transfer tool proximal hub has a central lumen, the proximal portion of the transfer sheath being disposed in the central lumen, the central lumen having a reduced diameter portion proximal to a proximal end of the transfer sheath. In some such embodiments, the transfer tool proximal hub also has a purge fluid port communicating with the central lumen and/or a seal adapted to seal around a catheter portion of a blood pump.

In some embodiments, the transfer tool further has a handle surrounding the transfer sheath. In some such embodiments, the handle extends proximally from the transfer tool connector. The transfer tool may also have a proximal hub, with the handle extending from the transfer tool connector to the proximal hub. The transfer tool connector may include threads disposed at a distal end of the handle (e.g., a rotatable ring with internal threads), the distal portion of the transfer sheath extending distally beyond the transfer tool connector.

In some embodiments wherein the distal portion of the transfer sheath is radially expandable.

Another aspect of the invention provides a method of deploying an expandable blood pump in a patient, the blood pump comprising an expandable and compressible pump housing, an impeller disposed in the pump housing, and a catheter extending proximally from the pump housing. In some embodiments, the method includes the steps of: moving at least a portion of the pump housing proximally into a transfer sheath of a transfer tool through a distal opening of the transfer sheath, the pump housing compressing as it enters the transfer sheath; advancing the transfer sheath distally into a hub of an introducer sheath disposed in a blood vessel of the patient; advancing the pump housing out of the transfer sheath into the introducer sheath; and advancing the pump housing out of the introducer sheath and into the blood vessel. In some embodiments, the transfer sheath advances distally into the hub of the introducer until a distal end of the transfer sheath abuts a proximal end of the introducer sheath. In some embodiments, the transfer sheath lumen has a diameter substantially equal to a diameter of the introducer sheath lumen.

Some embodiments include the step of connecting a connector of the transfer tool to a connector of the introducer hub. In some such embodiments, the connecting step includes the step of applying an axial force to move the transfer sheath and introducer sheath toward each other.

Some embodiments include the step of expanding the distal end of the transfer sheath as the pump housing moves into the transfer sheath. Some embodiments include the step of compressing the distal end of the transfer sheath before the distal end of the transfer sheath abuts the proximal end of the introducer sheath.

In some embodiments, the step of moving the pump housing proximally into the transfer sheath also includes the step of moving the pump housing proximally until proximal struts of the pump housing engage a sheathing stop at a proximal end of the transfer sheath. Some embodiments include the step of injecting purge fluid into a proximal end of the transfer sheath while the pump housing is disposed in the transfer sheath.

Yet another aspect of the invention provides a method of deploying an expandable blood pump in a patient, the blood pump comprising an expandable and compressible pump housing, an impeller disposed in the pump housing, and a catheter extending proximally from the pump housing. In some embodiments, the method includes the steps of: moving the pump housing proximally into a transfer sheath of a transfer tool through a distal opening of the transfer sheath, the pump housing compressing as it enters the transfer sheath and a distal end of the transfer sheath expanding as the pump housing enters the transfer sheath; advancing the transfer sheath distally into a hub of an introducer sheath disposed in a blood vessel of the patient; compressing the distal end of the transfer sheath within the hub; advancing the pump housing out of the transfer sheath into the introducer sheath; and advancing the pump housing out of the introducer sheath and into the blood vessel. In some embodiments, the transfer sheath lumen has a diameter substantially equal to a diameter of the introducer sheath lumen after the compressing step.

Some embodiments include the step of connecting a connector of the transfer tool to a connector of the introducer hub. In some such embodiments, the connecting step includes the step of applying an axial force to move the transfer sheath and introducer sheath toward each other.

Some embodiments include the step of compressing the distal end of the transfer sheath before the distal end of the transfer sheath abuts the proximal end of the introducer sheath. In some embodiments, the step of moving the pump housing proximally into the transfer sheath further includes the step of moving the pump housing proximally until proximal struts of the pump housing engage a sheathing stop at a proximal end of the transfer sheath.

Some embodiments include the step of injecting purge fluid into a proximal end of the transfer sheath while the pump housing is disposed in the transfer sheath.

Still another aspect of the invention provides a system for compressing a blood pump to a delivery configuration. In some embodiments, the system includes a transfer sheath having a lumen with a lumen diameter; and a sheathing tool engaged with a distal end of the transfer sheath, the sheathing tool having an inlet section and an outlet section, the inlet section having an inner surface defining a lumen whose diameter decreases from a distal end of the inlet section to a proximal end of the inlet section, the outlet section having a lumen extending from the inlet section to a proximal end of the sheathing tool so that it aligns with the transfer sheath lumen, the outlet section lumen having a diameter equal to or less than the transfer sheath lumen diameter, the sheathing tool being disengageable from the transfer sheath.

Some embodiments also include a fastener adapted to fasten the sheathing tool to the transfer sheath. In some embodiments, the transfer sheath has a splitting seam adapted to separate the transfer sheath into two of more pieces. In some embodiments, the transfer sheath has a hub on the proximal end of the transfer sheath. In some such embodiments, the hub has a distal face and a seal on the distal face. Alternatively or additionally, the hub may have a splitting seam adapted to separate the hub into two or more pieces.

Another aspect of the invention provides a method of loading a collapsible blood pump into a transfer sheath, the blood pump comprising a self-expandable housing, a self-expandable impeller disposed in the housing, a catheter extending proximally from the housing, and a drive shaft extending from the impeller through the catheter. In some embodiments, the method includes the steps of: pulling proximally on a portion of the catheter extending proximally from a proximal opening of the transfer sheath to move the housing proximally toward a distal opening of the transfer sheath; engaging the housing with a sloped surface of a sheathing tool disposed at the distal opening of the transfer sheath; compressing the housing and the impeller as the blood pump moves proximally with respect to the sloped surface into a lumen of the sheathing tool, the lumen of the sheathing tool having a diameter equal to or less than a diameter of a lumen of the transfer sheath; and pulling the housing into the transfer sheath lumen.

Some embodiments include the step of ceasing proximal pulling on the catheter when a distal end of the blood pump housing is disposed proximal to the distal opening of the transfer sheath. Some embodiments include the step of ceasing proximal pulling on the catheter when the self-expandable housing is compressed to the diameter of the transfer sheath lumen. Some embodiments include the step of removing the sheathing tool from the transfer sheath. Some embodiments include the step of connecting the catheter and drive shaft to a handle and a motor. Some embodiments include the step of extending the catheter and the drive shaft through the distal opening, the lumen, and the proximal opening of the transfer sheath.

Still another aspect of the invention provides a blood pump and delivery system having a collapsible blood pump with a self-expandable housing, an impeller disposed in the housing, a catheter extending proximally from a proximal end of the housing, and a drive shaft extending proximally from the impeller through the catheter, the collapsible blood pump having an expanded configuration in which the housing has an expanded outer diameter; a transfer sheath comprising a lumen having a distal opening, a proximal opening, and a lumen diameter less than the housing expanded outer diameter; a sheathing tool engaged with a distal end of the transfer sheath, the sheathing tool having an inlet section and an outlet section, the inlet section having an inner surface defining a lumen whose diameter decreases from a distal end of the inlet section to a proximal end of the inlet section, the outlet section having a lumen extending from the inlet section to a proximal end of the sheathing tool so that it aligns with the transfer sheath lumen, the outlet section lumen having a diameter substantially the same as the transfer sheath lumen diameter, the sheathing tool being disengageable from the transfer sheath; and an introducer sheath comprising a hub on a proximal end, a shaft extending distally from the hub and defining an introducer lumen having a diameter greater than an outer diameter of the transfer sheath, and a distal opening communicating with the introducer lumen, the introducer sheath shaft being configured to be inserted into a blood vessel of a patient.

Some embodiments also have a fastener adapted to fasten the sheathing tool to the transfer sheath. In some embodiments, the transfer sheath further has a splitting seam adapted to separate the transfer sheath into two of more pieces.

Some embodiments include a hub on the proximal end of the transfer sheath. In some such embodiments, the transfer sheath hub has a distal face and a seal on the distal face adapted to seal against a hub of the introducer sheath. The introducer sheath hub may also have a proximal face and a seal on the proximal face. In some embodiments, the transfer sheath hub has a splitting seam adapted to separate the hub into two or more pieces.

Some embodiments also include an unsheathing aid extending proximally from the transfer sheath hub, the unsheathing aid having a gripping tool adapted to enable a user to releasably grip the blood pump catheter and to move the catheter with respect to the transfer sheath. In some such embodiments, the gripping tool has a handle and a catheter engagement mechanism in the handle. The catheter engagement mechanism may include a movable button having an engaged position causing the handle to engage the catheter and a disengaged position in which the catheter can move with respect to the handle, and the button may be biased to the disengaged position by a spring. The unsheathing aid may also include a support shell surrounding the catheter, the support shell being disposed between the transfer sheath hub and the handle. In some such embodiments, the handle is movably supported by the support shell. The unsheathing aid may also be detachable from the transfer sheath hub.

In some embodiments, the hub includes a guide wire lumen adapted to receive a guide wire extending from the blood pump housing. Some such embodiments include a seal adapted to close the guide wire lumen.

In some embodiments, the hub has a sheath flush port in fluid communication with the introducer sheath. In some embodiments, the catheter extends proximally through the transfer sheath and through a proximal opening of the transfer sheath.

Some embodiments also include a guide wire extending proximally from the blood pump housing. In some such embodiments, the guide wire extends proximally through the transfer sheath and through a proximal opening of the transfer sheath.

In some embodiments, the introducer sheath shaft has a length extending distally from the introducer sheath hub that is less than a length of the transfer sheath extending distally from the transfer sheath hub. In some embodiments, the catheter and drive shaft are connectible to a handle and motor, and the system may include the handle and the motor.

Yet another aspect of the invention provides a method of deploying a blood pump within a patient, the blood pump comprising a self-expandable housing, an impeller disposed in the housing, and a catheter extending proximally from the housing. In some embodiments, the method includes the steps of: pulling proximally on a portion of the catheter extending proximally from a proximal opening of a transfer sheath to move the housing proximally toward a distal opening of the transfer sheath; compressing the housing and the impeller as the blood pump moves proximally into a lumen of the transfer sheath; inserting the transfer sheath through a proximal opening of an introducer sheath, the introducer sheath extending from its proximal opening disposed outside the patient distally into a blood vessel of the patient; advancing the transfer sheath within the introducer sheath until the distal opening of the transfer sheath is at or beyond a distal opening of the introducer sheath; and advancing the blood pump out of the transfer sheath, thereby permitting the blood pump housing and impeller to self-expand to an expanded configuration.

Some embodiments include the step of engaging the housing with a sloped surface of a sheathing tool disposed at the distal opening of the transfer sheath prior to the compressing step. The method may also include the step of disengaging the sheathing tool from the transfer sheath.

In some embodiments, the transfer sheath has a hub at a proximal end and the introducer sheath has a hub at a proximal end, the step of advancing the transfer sheath within the introducer sheath including the step of advancing the transfer sheath within the introducer sheath until the transfer sheath hub engages the introducer sheath hub. Some embodiments include the step of removing the transfer sheath from around the blood pump catheter after advancing the blood pump out of the transfer sheath.

In some embodiments, the blood vessel is a femoral artery and the introducer sheath has a length greater than or equal to 25 cm and less than or equal to 33 cm, the method further including the step of advancing the blood pump distally from a distal end of the introducer sheath to a pumping site in the patient's aorta in the expanded configuration. Some such embodiments also include the step of moving the blood pump in the expanded configuration proximally from the pumping site in the patient's aorta to the distal end of the introducer sheath. The method may also include the step of moving the blood pump proximally into the introducer sheath.

In some embodiments, the step of advancing the blood pump out of the transfer sheath includes the step of engaging the catheter with a gripping tool extending proximally from the transfer sheath and moving the catheter with respect to the transfer sheath.

Another aspect of the invention provides a catheter gripping tool. The device can comprise a base operably coupled to a depressible portion and one or more springs can be configured to bias the depressible portion away from the base, wherein the catheter gripping tool can be configured to regulate movement of a catheter during use.

In some examples, the one or more springs can comprise a plurality of torsion springs each having an aperture can be configured to engage an exterior surface of the catheter passing therethrough, wherein the depressible surface can regulate a diameter of each aperture from an engaged position to a disengaged position.

In some examples, the gripping tool can apply pressure to the exterior surface of the catheter to control a position of the catheter passing therethrough.

In some examples, the gripping tool can be configured to regulate movement of a sheathing catheter of an intravascular blood pump system.

In some examples, the device can be configured to slidably engage an exterior of a catheter, and wherein the catheter can be frictionally retained between the base and the depressible portion in an engaged configuration.

In some examples, the depressible portion can be configured to regulate a transition of the gripping tool from the engaged configuration to a disengaged configuration when a force can be applied against the depressible portion.

In some examples, the wherein the one or more springs can be configured to supply radial force against the catheter.

In general an intravascular blood pump system can comprise a catheter shaft extending through a lumen of a catheter, the catheter shaft having a proximal end and a distal end, the catheter shaft distal end operably coupled to an intravascular impeller locatable within a vessel, a catheter gripping tool in operable communication with the catheter, the catheter gripping tool comprising a base operably coupled to a depressible portion and one or more springs configured to bias the depressible portion away from the base, wherein the catheter gripping tool can be configured to regulate movement of the catheter, a handle comprising a sealed housing, a motor in operable communication with the catheter shaft proximal end, and a fluid pump operably coupled to the motor.

In some examples, the catheter shaft proximal end can be removably coupled to a rigid member extending through a sealed strain relief at a distal end of the handle.

In some examples, the system can further comprise a cradle assembly locatable around an exterior surface of the motor within the handle, the cradle assembly having one or more surfaces configured to retain a circuit board thereon.

In some examples, a cradle assembly comprises one or more routing channels configured to route one or more tubes within the sealed housing.

In some examples, the system may further comprise one or more conduits extending through a proximal strain relief configured to transition one or more electrical connections to a circuit board on the cradle assembly.

In some examples, the system can further comprise a catheter introducer having a proximal end configured to axially engage a sheath hub.

In some examples, the catheter can be a multi-layer catheter comprising at least one braid layer, the braid layer comprising one or more braid angles configured to conserve rotation from a proximal end of the multi-layer catheter to a distal end of the multi-layer catheter.

In some examples, the housing can be fluid-sealed.

In some examples, the system can further comprise one or more sensors operably connected to a circuit board locatable on the cradle assembly.

In some examples, the system can further comprise a pressure sensor configured to determine pressure of a fluid passing through the intravascular pump system.

In some examples, the system can further comprise a flow sensor configured to determine a flow rate of a fluid within the intravascular pump system.

In some examples, the system can further comprise a data transmission module configured to transfer data obtained within the system to one or more peripheral devices.

In some examples, the gripping tool comprises a depressible surface spring biased away from a base in a disengaged position.

In some examples, the system can further comprise a plurality of torsion springs each having an aperture configured to engage an exterior surface of the catheter passing therethrough, wherein the depressible surface regulates a diameter of each aperture from an engaged position to a disengaged position.

In some examples, the gripping tool applies pressure to the exterior surface of the catheter to control catheter movement.

In some examples, the gripping tool can be configured to regulate sheathing of an introducer as the intravascular blood pump system is advanced into a vessel.

In general, a method of operating a self-expanding blood pump within a patient can comprise the steps of inserting a compressed self-expanding blood pump within a transfer sheath through a proximal opening of an introducer sheath, the introducer sheath extending from its proximal opening disposed outside the patient distally into a blood vessel of the patient. Then, advancing the transfer sheath within the introducer sheath until a distal opening of the transfer sheath is at or beyond a distal opening of the introducer sheath. Then, advancing the self-expanding blood pump out of the transfer sheath to an expanded configuration at a deployment site within the blood vessel, wherein the self-expanding blood pump can be advanced by a catheter gripping tool in operable communication with an elongate member extending proximally from the self-expanding blood pump. Then, rotating one or more impellers of the self-expanding blood pump with a motor enclosed in a handle body, wherein a drive cable is operably coupled at a distal end to the one or more impellers and operably coupled at a proximal to the motor.

In some examples, the method can further comprise positioning the self-expanding blood pump within the blood vessel by rotating the handle body, wherein a drive cable catheter can be configured to rotate the self-expanding blood pump correspondingly to rotation of the handle body.

In some examples, the drive cable extends through a multi-layer drive cable catheter coupled to a distal end of the handle body, the multi-layer drive cable catheter configured to maintain even torque from the handle body to the self-expanding blood pump.

In some examples, the method can further comprise detecting a flow rate of a fluid from the blood pump to a motor assembly within the handle housing, wherein a one or more sensors are configured to detect the flow rate.

In general, a method of operating a self-expanding blood pump within a patient can comprise the steps of a gripping tool sheathing a self-expanding blood pump within a sheathing catheter. Then, advancing a distal end of the sheathing catheter through an introducer extending from outside the patient distally into a blood vessel of the patient. Then, advancing the sheathing catheter distal end into the blood vessel of the patient to a deployment site. Then, the gripping tool unsheathing the self-expanding blood pump to an expanded configuration at the deployment site within the blood vessel. Then, rotating one or more impellers of the self-expanding blood pump with a motor enclosed in a handle body, wherein a drive cable may be operably coupled at a distal end to the one or more impellers and operably coupled at a proximal to the motor.

These and other details and aspects are described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a collapsible blood pump and a first embodiment of a system for inserting the blood pump into a patient.

FIG. 2 shows the collapsible blood pump of FIG. 1 compressed inside a transfer sheath.

FIG. 3 shows the collapsible blood pump and transfer sheath of FIGS. 1 and 2 with a sheathing tool removed.

FIG. 4 shows the collapsible blood pump and transfer sheath of FIGS. 1-3 prior to insertion into an introducer sheath.

FIG. 5 shows the collapsible blood pump and transfer sheath of FIG. 4 inserted into the introducer sheath.

FIG. 6 shows the collapsible blood pump of FIG. 5 advanced out of the transfer sheath and expanded to its expanded configuration.

FIG. 7 shows the collapsible blood pump and introducer sheath of FIG. 6 with the transfer sheath removed.

FIG. 8 is an elevational view of portions of a collapsible blood pump, its transfer sheath and an introducer system according to a second embodiment of the invention.

FIG. 9 is a perspective view of a portion of the blood pump, transfer sheath and introducer system of FIG. 8.

FIG. 10 is a cross-sectional view of the blood pump, transfer sheath and introducer system of FIGS. 8 and 9.

FIG. 11 is a side view of an exemplary blood pump that includes an expandable scaffold that supports a housing with an impeller housed therein.

FIGS. 12A-H illustrate a method of loading a collapsible blood pump into an introducer sheath according to a third embodiment of the invention. FIG. 12A shows an introducer and a blood pump prior to loading the blood pump into a transfer tool. FIG. 12B shows the blood pump partially loaded into the transfer tool. FIG. 12C is a detail of the collapsible blood pump and a distal portion of a transfer sheath of the transfer tool as the pump is entering the transfer sheath. FIG. 12D shows the transfer tool engaged with an introducer. FIG. 12E shows the use of a gripping tool to advance the blood pump out of the introducer sheath. FIG. 12F shows the gripping tool in a retracted position after the blood pump has been advanced out of the introducer sheath. FIG. 12G shows the transfer tool retracted from the introducer sheath. FIG. 12H shows the collapsible blood pump retracted from the introducer sheath.

FIG. 13A is a cross-sectional view of a portion of the introducer of FIGS. 12A-H.

FIG. 13B is a partial cross-sectional view of the transfer tool of FIGS. 12A-H.

FIG. 13C is a detail cross-sectional view of a distal portion of the transfer sheath of FIG. 13B.

FIG. 14A is a perspective and partial cross-sectional view of a proximal portion of the introducer sheath of FIGS. 12A-H.

FIG. 14B is a partial cross-sectional view of a proximal portion of the transfer tool of FIGS. 12A-H.

FIG. 15A is a side elevational view of the transfer tool engaged with the introducer of FIGS. 12A-H.

FIG. 15B is a perspective view of the transfer tool engaged with the introducer of FIG. 15A.

FIG. 15C is a cross-sectional view of the transfer tool engaged with the introducer sheath of FIGS. 15A-B.

FIG. 15D is a partial cross-sectional view of the distal portion of the transfer tool engaged with the proximal portion of the introducer of FIGS. 15A-C.

FIG. 16 shows a catheter gripping tool in place on a catheter.

FIG. 17 is a side perspective view of the gripping tool shown in FIG. 16.

FIG. 18 is a rear view of the gripping tool shown in FIG. 16.

FIG. 19 is an exploded view of the gripping tool shown in FIG. 16.

FIG. 20 is a partially transparent view showing the gripping tool of FIG. 16 engaging a catheter.

FIG. 21 is a perspective view of the gripping tool of FIG. 16 with one or more components removed to show the tool's engagement with a catheter.

FIG. 22 is another perspective view of the gripping tool of FIG. 16 with one or more components removed to show the tool's engagement with a catheter.

FIG. 23 is yet another perspective view of the gripping tool of FIG. 16 with one or more components removed to show the tool's engagement with a catheter.

### DETAILED DESCRIPTION

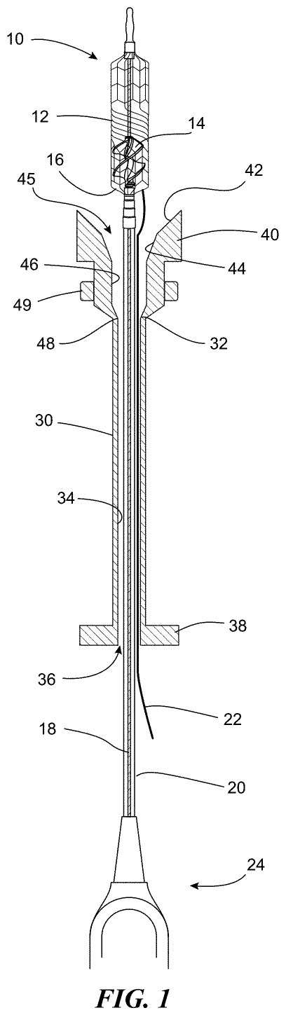

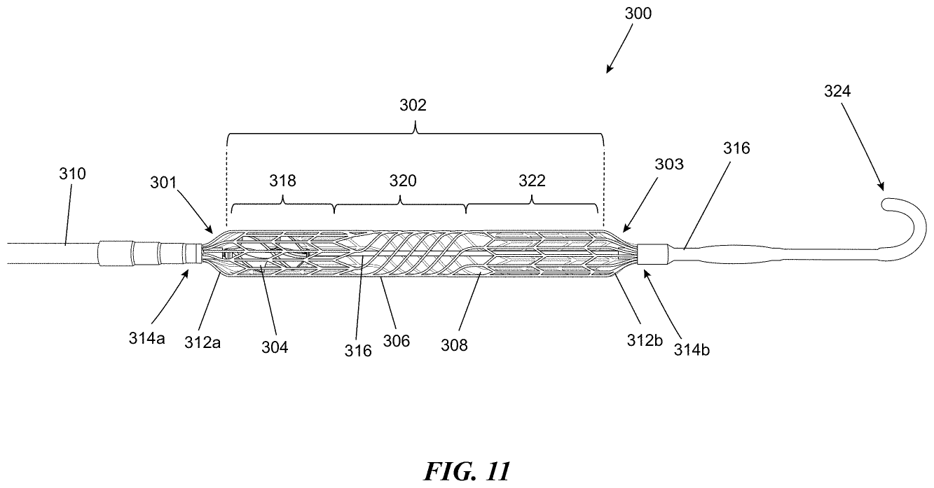

FIGS. 1-3 show a proximal portion of a blood pump 10 having a compressible and self-expandable outer housing 12, a rotatable compressible and self-expandable impeller 14 adapted to pump blood from an inlet (in a distal portion of the pump not shown in FIG. 1) through an outlet 16, and a drive shaft 18 extending proximally from the impeller through a catheter 20. An optional guide wire 22 extends proximally from the housing 12 along the outside of catheter 20, as shown, or alternatively through catheter 20. A handle 24 containing a motor (not shown) is connectable to catheter 20 and drive shaft 18 to operate the blood pump 10 by rotating the impeller 14. Housing 12 may also be referred to as a shroud. Alternatively, the invention may be used with blood pump 300 described below with respect to FIG. 11.

In order to more easily insert blood pump 10 into the vasculature of a patient (e.g., through an opening in a femoral artery) for advancement to a blood pump site (e.g., in the patient's heart and/or the patient's aorta), housing 12 and impeller 14 may be compressed to a smaller diameter delivery configuration with a transfer tool prior to insertion of the blood pump into the patient and optionally during advancement of the blood pump through the patient's vasculature after insertion. FIGS. 1-3 show steps of compressing the blood pump housing 12 as it is being inserted into a transfer sheath 30 of a transfer tool. Transfer sheath 30 may have a thin, flexible wall made of, e.g., silicone, high density polyethylene, polytetrafluoroethylene, and low density polyethylene. Prior to be being connected to handle 24 and the motor therein, catheter 20 and the drive shaft it contains may be passed into a distal opening 32, through an interior lumen 34, and out of a proximal opening 36 of transfer sheath 30.

The lumen 34 of transfer sheath 30 has an inner diameter less than the outer diameter of the blood pump housing 12 in its expanded configuration. To collapse blood pump housing 12 and place it within sheath 30, the catheter 20 of blood pump 10 is pulled proximally until housing 12 engages a first sloped surface 42 at an inlet section of a rigid sheathing tool 40 engaged with the distal opening 32 of sheath 30. Sloped surface 42 has an inner diameter defining a lumen 45 that decreases from the distal end of the sheathing tool proximally toward a constant diameter lumen 46 within an outlet section leading to a proximal opening 48 that lines up with the distal opening 32 and lumen 34 of transfer sheath. Lumen 46 has a diameter equal to or smaller than a diameter of the interior lumen 34 of transfer sheath 30. An optional second sloped surface 44 surrounding lumen 45 extends proximally from the first sloped surface 42 and is at a shallower angle with respect to the longitudinal axis of lumen 46.

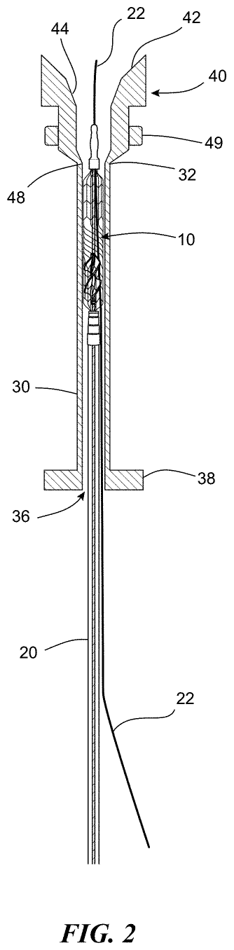

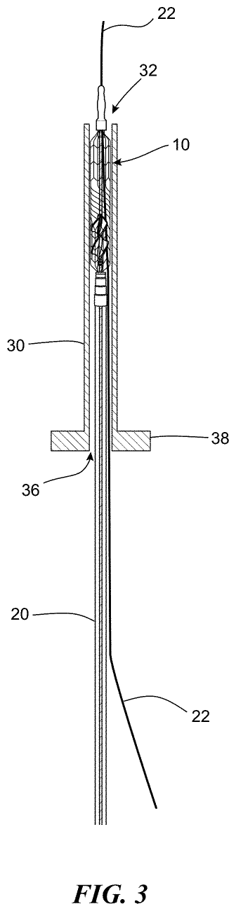

As blood pump 10 is pulled proximally with respect to sheathing tool 40, sloped surfaces 42 and 44 engage blood pump 10 to collapse housing 12 and impeller 14 to the collapsed delivery configuration shown in FIGS. 2 and 3. The proximal pulling on catheter 20 ceases when the distal end of blood pump 10 is at the distal opening 32 of transfer sheath 30. Alternatively, the proximal pulling on catheter 20 may cease when housing 12 has been compressed to the diameter of the transfer sheath lumen, even is the distal end of blood pump 10 is not yet inside of transfer sheath 30. The sheathing tool may then be removed, as shown in FIG. 3. In some embodiments, a sheathing tool fastener such as a ring 49 may be threaded or pressed on the exterior of the sheathing tool to compress it over the outside of the distal end of the transfer sheath 30 and removed to disengage the sheathing tool from the transfer sheath.

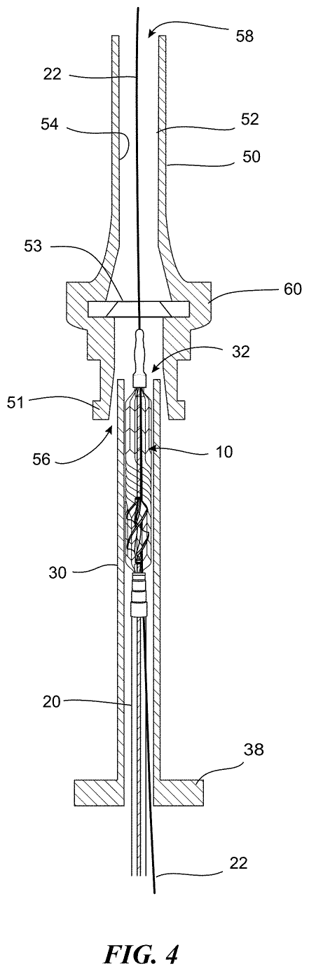

After the blood pump 10 has been loaded into transfer sheath 30, the transfer sheath 30 may be inserted into an introducer sheath 50 that has previously been inserted into the vasculature of the patient through, e.g., an opening in the femoral artery, as shown in FIG. 4. In some embodiments, introducer sheath 50 is 25-33 cm long and therefore extends only a short distance into the patient's vasculature. The introducer sheath 50 has a shaft 52 defining a lumen 54 extending between a proximal opening 56 and a distal opening 58. A hemostatic valve 53 may be disposed at the proximal opening 56. Lumen 54 has a diameter slightly greater than the outer diameter of transfer sheath 30. The proximal end 51 of shaft 52 may be flared to facilitate insertion of transfer sheath 30.

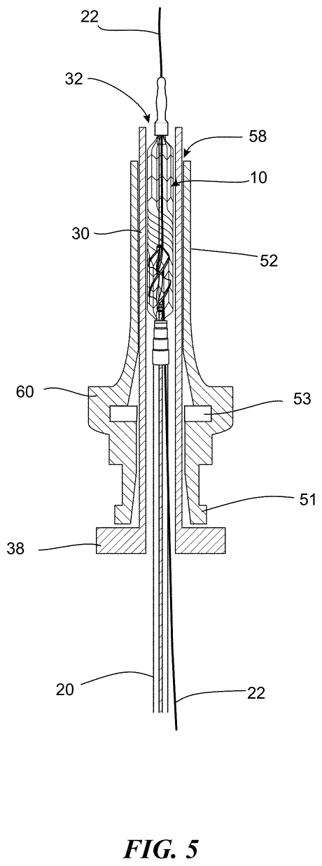

Transfer sheath 30 is advanced into introducer sheath 50 until a distal face of an optional hub 38 on the proximal end of transfer sheath 30 engages a proximal face of a hub 60 of introducer sheath 50. At this relative position of transfer sheath 30 and introducer sheath 50, blood pump 10 is at the distal opening 58 of introducer sheath 50, as shown in FIG. 5. The distal face of hub 38 and/or the proximal face of hub 60 may optionally have one or more seals. In some embodiments, such as the one shown in FIGS. 4-7, the shaft 52 of introducer sheath 50 may be shorter than transfer sheath 30 so that the distal end of transfer sheath 30 extends just beyond the distal end of introducer sheath 50 when the hubs 38 and 60 are engaged.

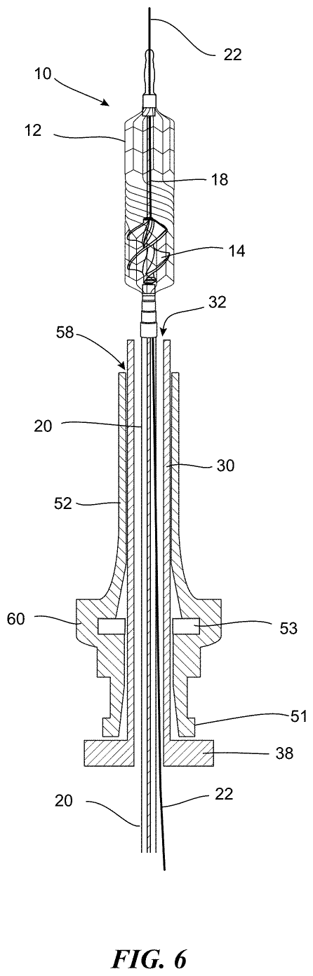

After hub 38 engages hub 60, any further distal movement of catheter 20 will advance blood pump 10 out of transfer sheath 30, thereby permitting it to self-expand to its expanded configuration as shown in FIG. 6. Blood pump 10 may then be advanced in its expanded configuration from the distal end of introducer sheath 50 through the patient's vasculature to the pumping site. The guide wire 22 may be managed by a single user as in a standard rapid exchange (Rx) device.

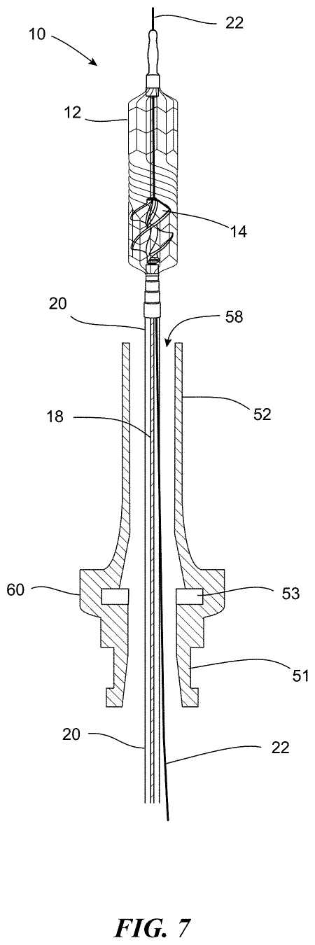

The transfer sheath 30 and its hub 38 may have optional splitting seams formed, e.g., as perforations that permit the sheath 30 to be separated into two or more pieces and removed from around the catheter 20 and guide wire 22 after the blood pump 10 has been advanced out of the sheath 30, as shown in FIG. 7. Removal of sheath 30 and hub 38 may not be necessary if one or more seals are provided between hub 38 and hub 60.

To remove blood pump 10 from the patient, blood pump 10 is withdrawn from the pumping site (e.g., in the aorta) in its expanded configuration to the distal end of the introducer sheath 50. Further retraction of blood pump 10 compresses it into the introducer sheath for removal from the patient.

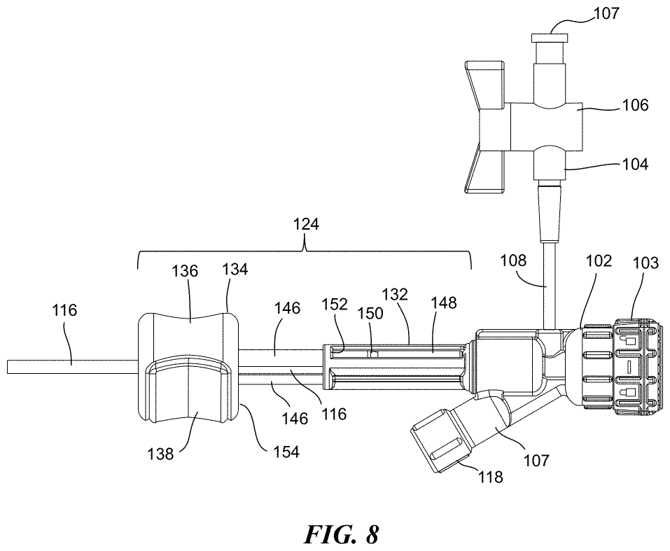

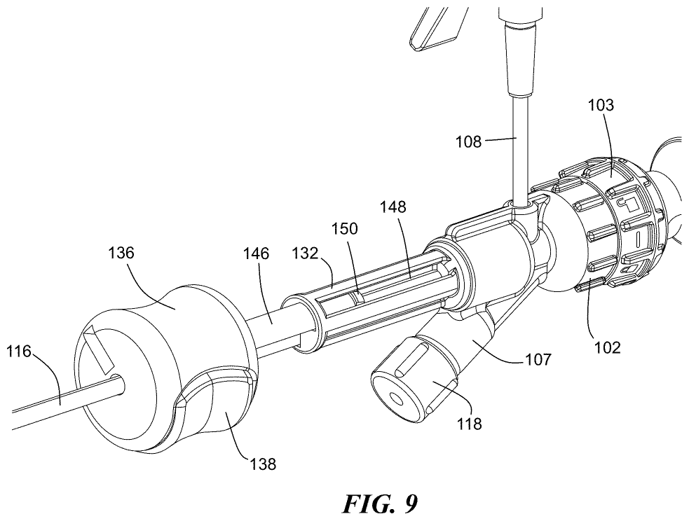

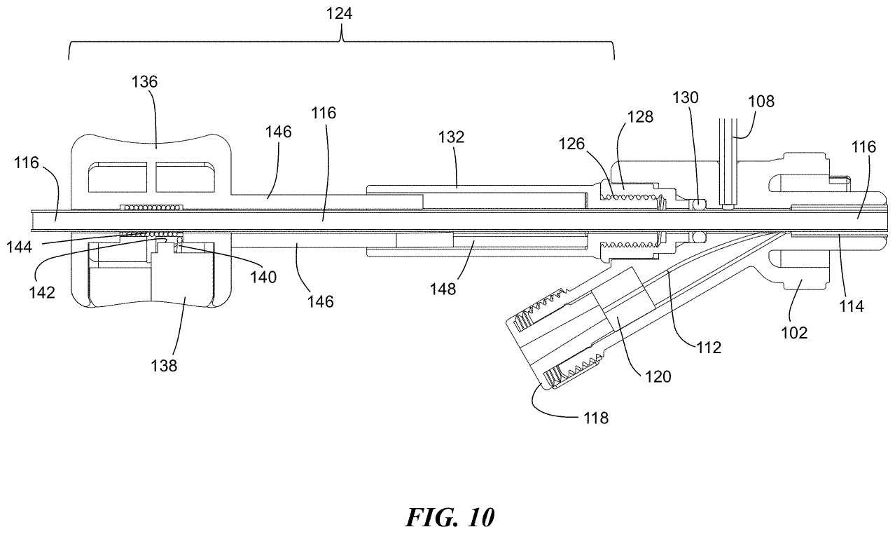

FIGS. 8-10 show portions of a collapsible blood pump, a transfer tool and an introducer system according to an embodiment of the invention. The transfer sheath 100 of the transfer tool of this embodiment is bonded to a hub 102 that interlocks with the hub 103 of an introducer sheath (not shown) extending distally from hub 103. The hub 102 may connect to a sheath flush mechanism 104 with a valve 106, inlet port 107, and conduit 108 to be used to deliver saline or other flushing fluid to the introducer sheath. A side arm 110 has a guide wire lumen 112 leading to an annular space between the transfer sheath 114 into which a collapsible blood pump (not shown) has been loaded (e.g., as discussed above) and the catheter 116 of the collapsible blood pump. The guide wire (not shown) of the collapsible blood pump within the transfer sheath 114 is disposed in guide wire lumen 112 when the pump is placed in the transfer sheath. A threaded luer cap 118 may be used to compress a seal 120 to close the guide wire lumen 112.

Catheter 116 extends proximally from a collapsible blood pump (not shown) that is disposed within transfer sheath 100. As discussed above, in some embodiments the collapsible blood pump may be disposed at a distal opening of the transfer sheath. Advancement of the blood pump with respect to the transfer sheath, e.g., by pushing on the catheter extending proximally from the blood pump, will permit the collapsed blood pump to emerge from the distal end of the transfer sheath and expand to its expanded configuration. To aid with the advancement and deployment of the blood pump, an unsheathing aid 124 is detachably attached to a proximal end of hub 102 via, e.g., threads 126 that engage corresponding threads of an insert 128. An O-ring seal 130 is disposed between insert 128 and hub 102.

Extending proximally from the threads 126 of unsheathing aid 124 is an annular support shell 132 surrounding catheter 116 that prevents the catheter from kinking while it is being advanced. A translatable gripping tool 134 is attached to support shell 132 via gripping tool arms 146 that slide within open grooves 148 of support shell 132. Tabs 150 extending radially outward from a distal end of arms 146 engage distally facing surfaces 152 of open grooves 148 to limit proximal movement of gripping tool 134 with respect to support shell 132. Distal movement of gripping tool 134 is limited by engagement of a distal face 154 of a handle 136 with a proximally facing surface 156 of support shell 132.

Handle 136 (optionally disposed at a proximal end of the gripping tool, as shown) supports a depressible button 138. A spring 140 biases button 138 in the disengaged position shown in FIGS. 8-10. When depressed against the bias of spring 140, a surface 142 on the underside of button 138 engages a torsion spring 144 surrounding catheter 116, causing spring 144 to grippingly engage catheter 116. Releasing button 138 allows spring 140 to move surface 142 away from spring 144, thereby releasing the grip on the catheter.

Advancing gripping tool 134 distally with respect to support shell 132 while button 138 is depressed and torsion spring 144 is engaged with catheter 116 advances catheter 116 and the blood pump distally with respect to the transfer sheath 100. In some embodiments, a single advancement of gripping tool 134 when engaged with catheter 116 until handle 136 engages the support shell will advance the compressed blood pump completely out of the transfer sheath. In other embodiments, the catheter will have to be advanced and released by the gripping tool 134 two or more times in order to move the blood pump out of the transfer sheath. In still other embodiments, the catheter may be provided with a mark that, e.g., lines up with handle 136 when the catheter has been advanced far enough to push the blood pump out of the transfer sheath. Tactile feedback to the user will also indicate when the blood pump has completely emerged from the transfer sheath; the frictional forces between the collapsed blood pump and the transfer sheath will disappear, and advancement will become much easier, when the blood pump is out of the transfer sheath.

The unsheathing aid 124 may also be used to resheathe the blood pump. As the catheter 116 and blood pump are withdrawn proximally and engage the distal opening of the introducer sheath, the gripping tool can be used to grab and pull the catheter into the introducer sheath.

FIG. 11 shows a side view of an exemplary intravascular catheter blood pump 300. The blood pump 300 includes an expandable/collapsible blood conduit or housing 302 that is configured to transition between an expanded state, as shown in FIG. 11, and a collapsed state (not shown). For example, the housing 302 may be in the collapsed state when confined within an introducer sheath or a delivery catheter for delivery to the heart, expanded upon release from the introducer sheath or delivery catheter for blood pumping, and collapsed back down within the introducer sheath or the delivery catheter (or other catheter) for removal from heart. When in the expanded state, the housing 302 is radially expanded so as to form an inner lumen for passing blood therethrough. When in the expanded state, the inner lumen of the housing 302 may be configured to accommodate blood pumped by one or more impellers therein. The one or more impellers may be collapsible so that they may collapse to a smaller diameter when the housing 302 is in the collapsed state. The one or more impellers may be positioned within one or more impeller regions of the housing 302. In some examples, the impeller region(s) of the housing 302 is/are radially stiffer than other regions (e.g., adjacent regions) of the housing 302 to prevent the impeller(s) from contacting the interior walls of the housing 302.

In this example, the blood pump 300 includes an impeller 304 within a proximal portion of the housing 302. In some cases, the blood pump 300 can include more than one impeller. For example, the blood pump 300 may include a second impeller in a distal region 322 of the housing 302. In some cases, blood pump 300 may include more than two impellers. The housing 302 includes a first (e.g., proximal) end having first (e.g., proximal) openings 301, and a second (e.g., distal) end having second (e.g., distal) openings 303. The first openings 301 and second openings 303 may be configured as and an outlet and inlet, respectively, for blood pumped by blood pump 300. For example, blood may largely enter the housing 302 via the second (e.g., distal) openings 303 and exit the housing 302 via the first (e.g., proximal) openings 301. In such case, the second openings 303 act as a blood inlet and the first openings 301 act as a blood outlet. The one or more impellers (e.g., impeller 304) may be configured to pump blood from the inlet toward the outlet. In an exemplary operating position, the second openings 303 (e.g., inlet) may be distal to the aortic valve, in the left ventricle, and the first openings 301 (e.g., outlet) may be proximal to the aortic valve (e.g., in the ascending aorta).

The housing 302 includes a tubular expandable/collapsible scaffold 306 that provides structural support for a membrane 308 that covers at least a portion of inner surfaces and/or outer surfaces of the scaffold 306. The scaffold 306 includes a material having a pattern of openings with the membrane 308 covering some or all of the openings (other than first openings 301 and second openings 303) to channel the blood through the lumen of the housing 302. The scaffold 306 may be unitary and may be made of a single piece of material. For example, the scaffold 306 may be formed by cutting (e.g., laser cutting) a tubular shaped material. Exemplary materials for the scaffold 306 may include one or more of: nitinol, cobalt alloys, and polymers, although other materials may be used.

The blood pump 300 includes proximal struts 312a that extend from the scaffold 306 and at least partially defining the first openings 301 (e.g., blood outlet region) and distal struts 312b that extend from the scaffold 306 and at least partially define the second openings 303 (e.g., blood inlet region). The proximal struts 312a are coupled to first hub 314a of a proximal shaft 110. The distal struts 312b are coupled to second hub 314b of a distal portion 314. In this example, the first hub 314a includes a bearing assembly through which a central drive cable 316 extends. The drive cable 316 is operationally coupled to and configured to rotate the impeller 304.

In some cases, the impeller 304 is fully positioned axially within the housing 302. In other cases, a proximal portion of the impeller 304 is positioned at least partially outside of the housing 302. That is, at least a portion of the impeller may be positioned in axially alignment with a portion of the struts 312a and openings 301.

The housing 302 and the scaffold 306 may characterized as having a proximal region 318, a central region 320, and a distal region 322. The central region 320 may be configured to be placed across a valve (e.g., aortic valve) such that the proximal region 318 is at least partially within a first heart region (e.g., ascending aorta) and the distal region 322 is at least partially within a second heart region (e.g., left ventricle). The proximal region 318 (and in some cases the distal region 322) may be configured to house an impeller therein. The proximal region 318 may (and in some cases the distal region 322) has a stiffness sufficient to withstand deformation during operation of the blood pump 300 when within the beating heart and to maintain clearance (i.e., a gap) between an impeller region of the blood pump 300 and the rotating impeller 304. The distal region 322 includes the second (e.g., distal) opening 303 of the housing 302, and may serve as the blood inlet for the housing 302.

The central region 320 may be less rigid relative to the proximal region 318 (and in some cases the distal region 322). The higher flexibility of the central region 320 may allow the central region 320 to deflect when a lateral force is applied on a side of the housing 302, for example, as the housing 302 traverses through the patient's blood vessels and/or within the heart. For example, the central region 320 may be configured to laterally bend upon a lateral force applied to the distal region 322 and/or the proximal region 318. In some cases, it may be desirable for the central region 320 to laterally bend as the housing 302 traverses the ascending aorta and temporarily assume a bent configuration when the housing 302 is positioned across an aortic valve. In this example, the central region 320 includes a helical arrangement of longitudinally running elongate elements configured to provide flexibility for lateral bending. In some examples, a distal tip 324 of the blood pump 300 is curved to form an atraumatic tip. In some cases, the distal tip 324 flexible (e.g., laterally bendable) to enhance the atraumatic aspects of the distal tip 324. For example, the distal tip 324 may be sufficiently flexible to bend when pressed against tissue (e.g., by a predetermined amount of force) to prevent puncture of the tissue.

The first hub 314a (e.g., proximal hub) and/or the second hub 314b (e.g., distal hub) may include features that promote smooth blood flow into and/or out of the housing 302. Such features may prevent or reduce the occurrence of stagnant and/or turbulent blood flow that may otherwise tend to occur in regions near the first opening 301 (e.g., outlet region) and/or the second opening 303 (e.g., inlet region) of the housing 302. Since stagnant and/or turbulent blood flow is associated with blood coagulation and/or clotting, measures to reduce this can be beneficial to for patient outcome.

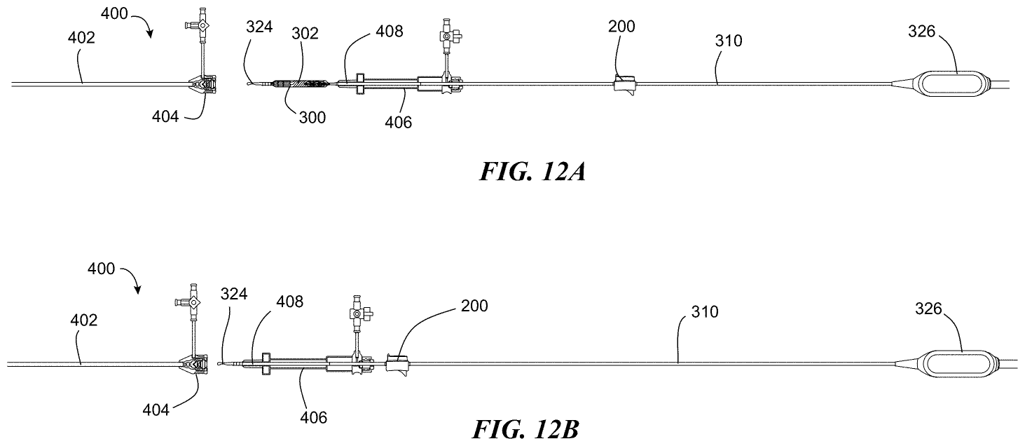

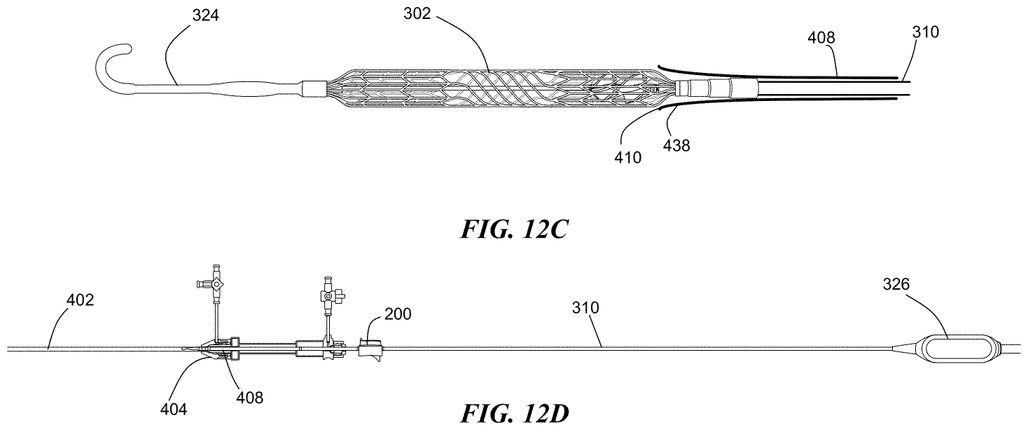

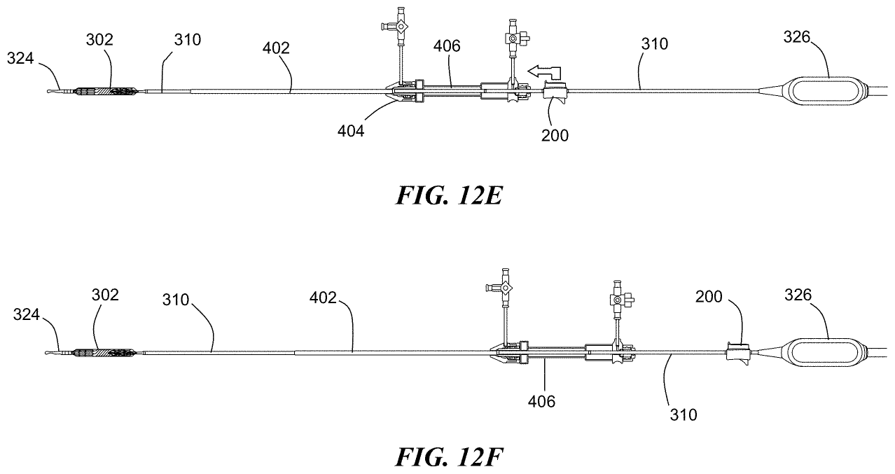

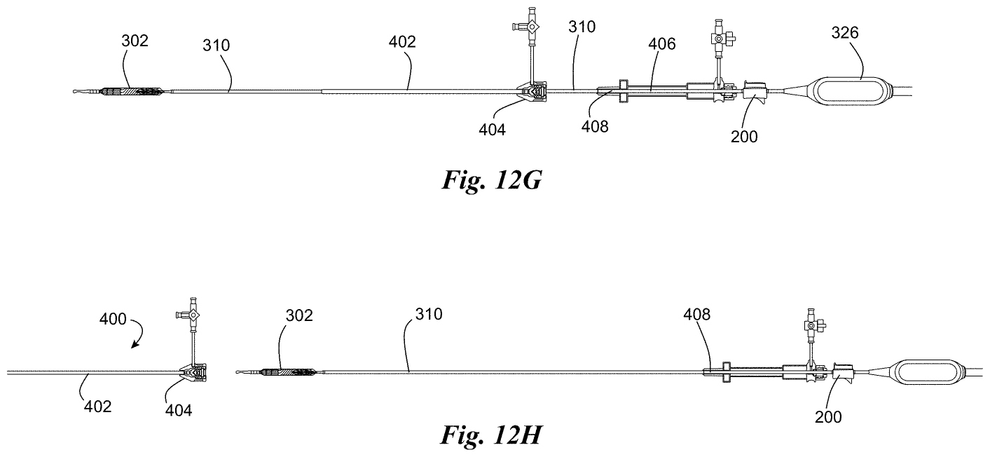

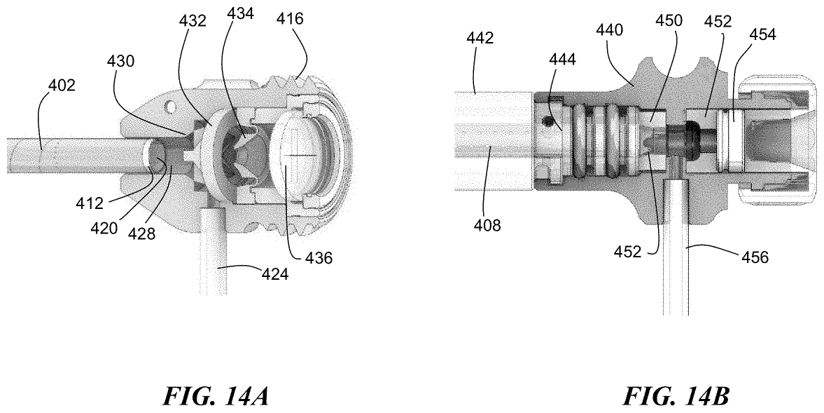

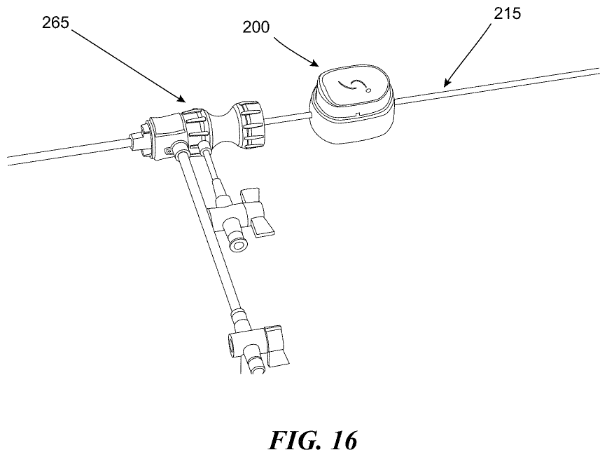

FIGS. 12A-15D show another embodiment of a system and method for inserting a collapsible blood pump into a patient, such as pump 300 illustrated in FIG. 11. The system includes an introducer 400 having an introducer sheath 402 and an introducer hub 404. The system also includes a catheter gripping tool 200 (such as the gripping tools described above with respect to FIGS. 8-10 and below with respect to FIGS. 16-23) and a transfer tool 406 having a transfer sheath 408.

A method of using the system to introduce blood pump 300 into a patient is illustrated in FIGS. 12A-H. FIG. 12A illustrates the blood pump and insertion system as supplied to the user. As shown, blood pump 300 has an expandable/collapsible housing 302 proximal to a distal tip 324. A catheter 310 extends proximally from housing 302 to a handle 326 housing, e.g., a motor for operating the impeller disposed within housing 302. Surrounding catheter 310 just proximal to expandable/collapsible housing 302 is the transfer sheath 408 of transfer tool 406. Catheter gripping tool 200 is engaged with catheter 310 at a location proximal to transfer tool 406. Blood pump 300 has not yet been placed within transfer tool 406 or introducer 400.

A possible target deployment location for the housing 302 (and impeller) portion of blood pump 300 is a position extending from the patient's aorta into the left ventricle of the patient's heart. When deployed at this target location, the catheter 310 extends proximally from housing 302 through the patient's vasculature to handle 326, which remains outside of the patient's body. To reach this target location, the housing 302 and the distal portion of catheter 310 must be advanced through the patient's vasculature from an entry point, such as an incision in the patient's femoral artery. Introducer 400 may be inserted through the entry point into the femoral artery to provide a lumen through which the blood pump may be inserted and advanced.

Because the femoral artery has a much smaller diameter than the aorta, the interior lumen 420 of introducer sheath 402 has a diameter smaller than the expanded diameter of housing 302. Housing 302 is therefore collapsed by transfer tool 406 prior to insertion of the housing into the introducer sheath 402. As shown in FIGS. 12B and 12C, pulling proximally on catheter 310 while holding transfer tool 406 stationary draws housing 302 proximally into the distal end 410 of transfer sheath 408, which has a diameter less than the expanded diameter of housing 302. As shown in FIG. 12C, the atraumatic distal portion 438 of transfer sheath 408 expands, and housing 302 collapses, as housing 302 enters transfer sheath 408. The impeller within housing 302 may also collapse as housing 302 enters transfer sheath 408.

An engageable and disengageable catheter gripping tool, such as the gripping tool 200 described below with respect to FIGS. 16-23 or gripping tool 134 described above with respect to FIGS. 8-10, may be used to advance or retract catheter 310. For example, gripping tool 200 may be disengaged from catheter 310 and advanced from the position shown in FIG. 12A to the position shown in FIG. 2B, then reengaged with catheter 310. Gripping tool 200 may then be used to pull catheter 310 further proximally to move housing 302 into sheath 408. The disengagement of gripping tool 200, movement of gripping tool 200 with respect to catheter 310, reengagement of gripping tool 200 with catheter 310, and joint proximal movement of gripping tool 200 and catheter 310 may be repeated until housing 302 is entirely within sheath 408.

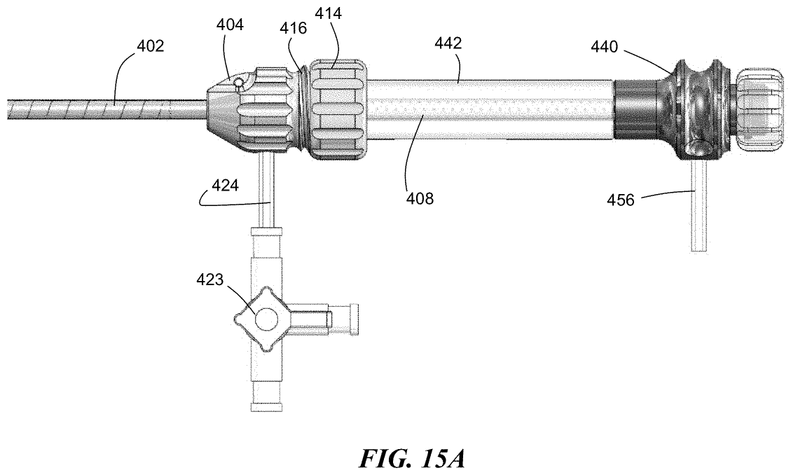

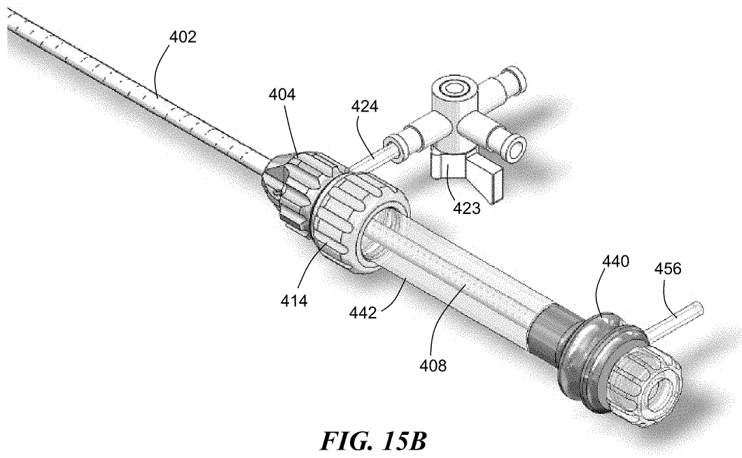

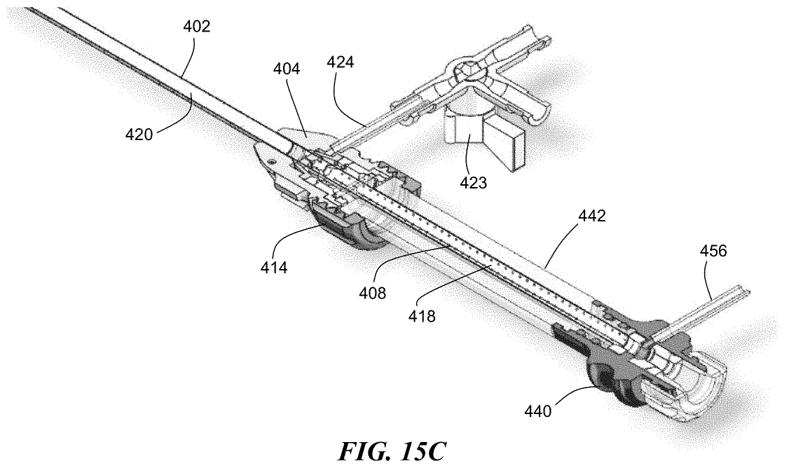

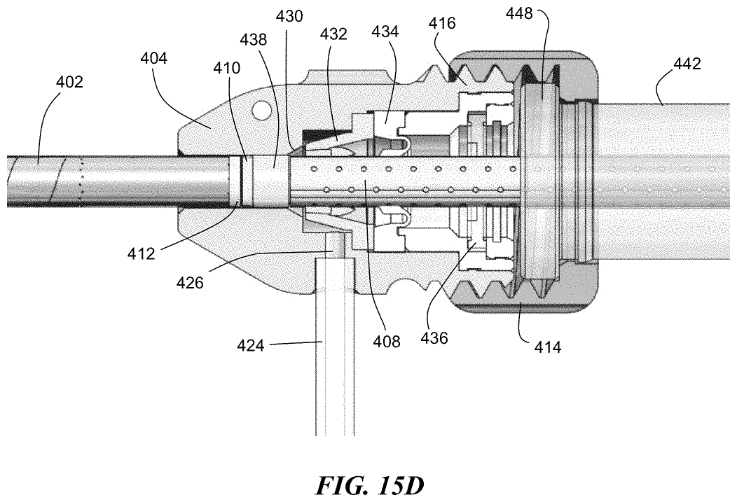

After the collapsible portion of blood pump 300 is entirely within sheath 408, the transfer tool 406 is advanced toward the introducer 400, the sheath 402 of which has already been inserted into the patient's femoral artery (or other access point) with introducer hub 404 remaining outside the patient. The distal end of transfer sheath 408 is inserted into introducer hub 404 until the distal end 410 of transfer sheath meets the proximal end 412 of introducer sheath 402, as shown in FIG. 12D. As shown in FIGS. 15A-D, a transfer tool connector 414 may connect to a corresponding connector 416 on introducer hub 404 when the distal end 410 of transfer sheath 408 meets the proximal end 412 of introducer sheath 402 to lock the two elements in place.

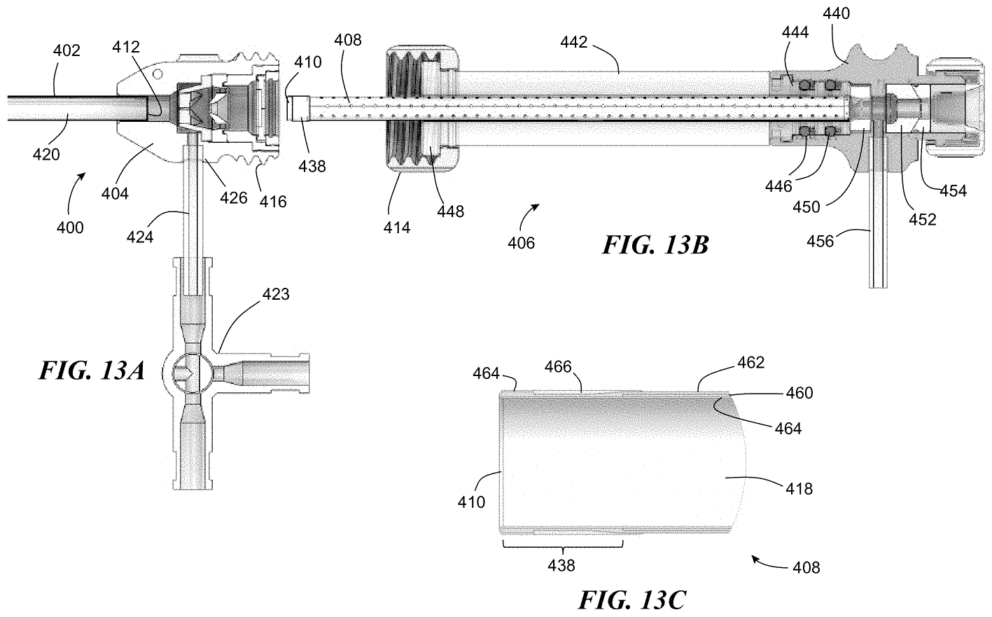

In order to promote smooth movement of the collapsed blood pump out of transfer sheath 406 into introducer sheath 402, the internal diameters of the transfer sheath lumen 418 and the introducer sheath lumen 420 may be substantially equal when the distal end 410 of transfer sheath 408 meets the proximal end 412 of the introducer sheath 402. The internal lumen of the introducer hub 404 may have a chamfered or sloped surface 430 (shown in FIGS. 13A, 14A, and 15D) that engages the distal of the transfer sheath 408 as the transfer sheath moves into the introducer hub 404 to reduce the previously-expanded diameter of the distal end 410 of the transfer sheath 408 as it meets the proximal end 412 of introducer sheath 402.

After the transfer tool 406 has been engaged with, and locked to, the introducer 400, the expandable portion of the blood pump may be advanced out of the transfer sheath 406 into the introducer sheath 402, and then from the introducer sheath 402 into and through the patient's vasculature to the target deployment site, as shown in FIGS. 12E-F. The gripping tool 200 may be repeatedly used to grip the catheter for advancement, disengaged from catheter 310 for movement to a more proximal position on the catheter, and reengaged with the catheter for further gripping and advancement of the catheter until the expandable housing 302 has reached the target deployment site. The transfer tool 406 and gripping tool 200 may then be moved proximally toward the blood pump handle 326, as shown in FIG. 12G, to reduce the load at the incision site. After completion of the blood pumping procedure, the blood pump may be removed from the patient by pulling catheter 310 proximally (possibly with gripping tool 200) through introducer sheath 402, as shown in FIG. 12H. A distal portion of introducer sheath 402 may expand as the blood pump housing enters to help collapse the housing into the sheath, as described below.

FIGS. 13A-15D show further details of the introducer and transfer tool. Introducer sheath 402 is a flexible composite shaft formed from a laser cut hypotube 460 with a PTFE liner 464 and a ChronoFlex® thermoplastic urethane outer jacket 462 reflowed through the laser cut openings for bonding to the hypotube. Introducer sheath may be, e.g., 24 cm long, and it may have an expandable and atraumatic distal tip, as described below with respect to FIG. 13C. A stopcock 423 and fluid line 424 (formed, e.g., from Tygon® tubing) lead to fluid port 426 in hub 404 to provide, e.g., a purge fluid inlet for use during a blood pumping procedure or for aspiration.

Hub 404 has an internal lumen extending therethrough. The proximal end of introducer sheath 402 is disposed in a distal portion 428 of the hub lumen. A chamfered surface 430 is disposed at the proximal end of distal lumen portion 428 to guide and compress the distal end 410 of transfer sheath 408 as it is inserted into distal lumen portion 428 of introducer hub 404.

A one-way valve 432 (e.g., a cross-slit duckbill valve) is disposed in a larger diameter portion of the introducer hub lumen proximal to chamfered surface 430. Valve 432 seals against vascular pressure prior to insertion of the transfer sheath and pump into introducer 400. Valve 432 is configured to maintain sealing functionality after long-term use as it closes as a result of back-pressure and not as a result of elasticity of the material. Valve 432 can be constructed from an elastomeric material with a durometer that can range from approximately 25 A-90 A. In some examples the valve can be made from 50 A Silicone. A radial seal 434 (e.g., a silicone aperture valve) is disposed in the introducer hub lumen proximal to duck-bill valve 432. Seal 434 can accommodate a range of diameters of devices inserted through it (e.g., 1 mm-6 mm diameter). Seal 434 seals around the transfer sheath 406 and catheter 310 when inserted. In the illustrated embodiment, seal 434 is convoluted to maintain a seal even if the sheath or catheter is inserted through it on an angle with respect to the longitudinal axis of the hub. Radial seal 434 can be constructed from an elastomeric material with a durometer that can range from approximately Shore 25 A-90 A. In some examples, the valve can be made from a silicone material with a high degree of elongation. An elastomeric disc valve 436 is disposed in the introducer hub lumen proximal to radial seal 434 to provide an additional seal around any guidewire that is outside the catheter and around the catheter itself. In the illustrated embodiment, valve 436 consists of an elastomeric material that has a thickness from approximately 0.1 mm-10 mm. The durometer can range from approximately Shore 25 A-90 A. It may contain a plurality of cross cuts on each face on either side of the thickness that extend through at least a portion of the thickness. In some examples the valve can be a silicone disc made from 30 A durometer material and has a cross-cut on each face oriented to each other at 90 degree angles.

In some embodiments, the valves and seals are ordered from distal to proximal: one-way valve 432, radial seal 434, disc valve 436. In another embodiment, one-way valve 432 may not be present and only radial seal 434 and disc valve 436 are used. In another embodiment, disc valve 436 alone is used. In another embodiment, one-way valve 432 and radial seal 434 are disposed within the introducer hub, and disc valve 436 is disposed within a separate, removeable component.

The introducer hub connector 416 has a connector configured to adjoin to the transfer sheath connector. This connector may contain a twist-lock bayonet feature, a living hinge snap fit, a spring-loaded engagement, and/or internal/external threads. These connecting features may serve to align and provide an axial force to draw the distal end of the transfer sheath tip towards the proximal end of the introducer shaft. In the illustrated embodiment, introducer hub connector 416 is an exterior threaded surface on the proximal end of introducer hub 404, and the transfer tool connector 414 is a rotatable ring with internal threads. In this embodiment, when transfer tool connector 414 is threaded onto introducer hub connector 416, the distal end of transfer sheath advances into introducer hub lumen to place the distal end 410 of transfer sheath 408 into contact with the proximal end 412 of introducer sheath 402, as shown in FIG. 15D.

Transfer sheath 408 consists of a tube with sufficient column strength to sheath the pump housing. Sheath 408 may consist of a tube made from a rigid and/or lubricious polymer such as PTFE, or may be a composite construction. In some embodiments a composite construction consists of laser-cut metal tube in which the laser cut pattern is tailored for flexibility and/or rigidity in desired regions. In some embodiments, the laser-cut metal tube can be lined with a lubricous polymer, and jacketed with a polymer to adhere to the liner. In one embodiment, transfer sheath 408 is a composite shaft formed from a rigid laser cut hypotube 460 with a PTFE liner 464 and a ChronoFlex® thermoplastic urethane outer jacket 462 reflowed through the laser cut openings to the hypotube 460 and to the outer surface of the PTFE liner 464. A flexible distal portion 438 at the distal end of transfer sheath 408 flares as the housing of the blood pump enters the sheath, as shown schematically in FIG. 12C, to minimize damage to the blood pump while the housing is compressed within transfer sheath 408.

FIG. 13C shows details of distal portion 438. Laser cut hypotube 460 is covered by thermoplastic urethane outer jacket 462, which extends beyond the distal end of hypotube 460 into distal portion 438. PTFE liner 464 extends through hypotube 460, through the portion of outer jacket 462 extending beyond hypotube 460, and around the distal end of outer jacket 462 to cover the distal end of outer jacket 462. A flexible ring 466 (made, e.g., of Pebax® elastomer) is disposed on the outer surface of jacket 462 proximal to the external portion of liner 464, then reflowed with the thermoplastic of the outer jacket to shape and attach the elements together. The elastomeric properties of ring 466 enable distal region 438 to expand as the pump housing is pulled into sheath 408 and to collapse the pump housing without damaging the pump. This same expandable and atraumatic structure (the outer jacket extending beyond the end of the hypotube, liner extending around the end of the outer jacket, an elastomeric ring on the outside of the outer jacket) may be employed at the proximal end of transfer sheath 408 and on the distal and proximal ends of introducer sheath 402.

The proximal end of transfer sheath 408 is disposed in the proximal section 444 of a transfer tool housing 442 within a transfer tool hub 440. Silicone O-rings 446 disposed between the housing 442 and hub 440 provide a seal against vascular pressure. Housing 442 extends from hub 440 and transfer tool connector 414 to provide a grip for relative movement between the blood pump and the transfer tool. A raised edge 448 at the distal end of housing 442 provides a surface around which connector 414 rotates.

A sheathing stop 450 provides a proximal limit to movement of the pump housing during sheathing, and it protects the struts at the proximal end of the housing from damage. Sheathing stop 450 also provides a smooth guidewire lead-in and transition during guidewire loading. In some embodiments, sheathing stop 450 is formed from a molded or machined polymer and is configured to be atraumatic to the pump legs and guidewire. This can be achieved either as a result of the material selection or internal geometry. In one embodiment sheathing stop 450 is formed from ChronoFlex® thermoplastic urethane and is disposed within hub 440 proximal to the proximal section 444 of housing 442. In one embodiment, sheathing stop 450 has a tapered internal lumen 452 whose diameter decreases from its distal end toward its proximal end.

A seal 452 (e.g., a Tuohy-Borst seal) provides a seal around the blood pump catheter as it extends proximally through the transfer tool. A molded polymer spacer 454 provides a nominal compression of the Tuohy-Borst seal to minimize the amount of adjustment of the Tuohy-Borst seal required to be made by a user. Spacer 454 also provides a lead-in for the guidewire. A fluid line 456 (formed, e.g., from Tygon® tubing) leads to a fluid port through hub 440 to provide, e.g., a purge fluid access to the interior lumen of transfer sheath 408. For example, while loading a blood pump into transfer tool 406, saline fluid can be supplied through fluid line 456 to displace air within the transfer sheath and/or blood pump. When the transfer tool is connected to the introducer, saline fluid can be supplied to the introducer through fluid line 424 to introducer sheath 402.

FIGS. 16-23 illustrate another embodiment of a catheter gripping tool that can be used as part of the system for inserting a collapsible blood pump into a patient described herein. FIG. 16 shows gripping tool 200 in operable communication with an elongate member 215 (e.g., a catheter or sheath), such as the blood pump catheter described above. In this example, the gripping tool is positioned proximal to an introducer hub 265 and can be configured to advance, rotate, retract, or otherwise manipulate the elongate member (e.g., a catheter) passing through the hub 265. For example, a blood pump system, as described herein can have a drive cable extending through a drive cable catheter and the drive cable catheter can be an elongate member controllable with the gripping tool. In such an example, the gripping tool can apply gripping force to the drive cable catheter to advance, retract, rotate, or otherwise manipulate the drive cable catheter, which can translate to manipulation to one or more structures distal or proximal on the elongate member.

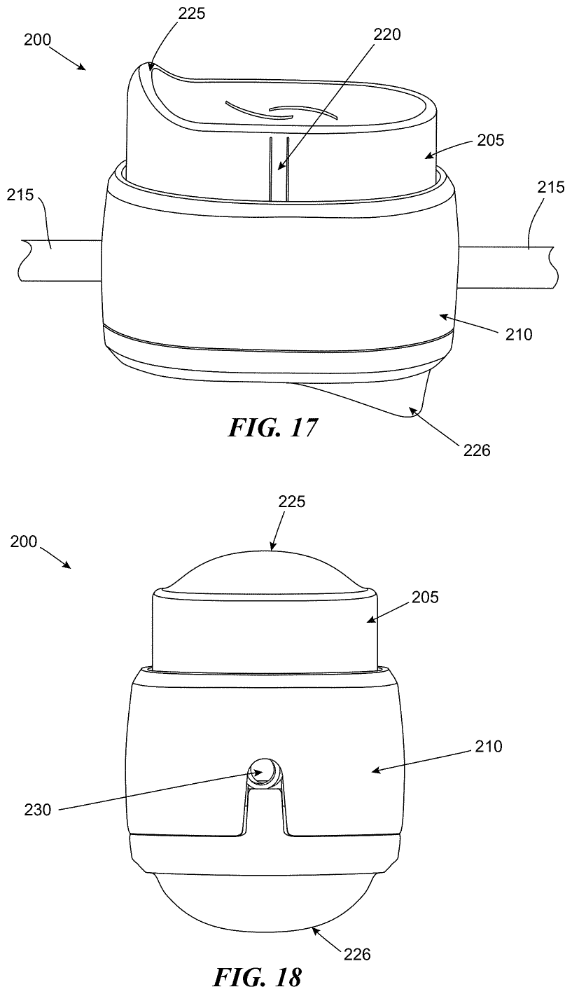

As shown in FIG. 17, gripping tool 200 has a housing with an activation element 205 and a body portion 210. An illustrative section of a catheter 215, such as the proximal shaft 110 of blood pump 100 illustrated in FIG. 11, is shown extending through the gripping tool 200. In some examples, the activation element 205 is configured to operate a retention element (not shown in FIG. 17) within the gripping tool. For example, the activation element 205 is shown in FIG. 17 as a depressible element (e.g., a button). The activation element 205 may have one or more external alignment features 220 (e.g., channels) configured to engage a corresponding alignment element of the body portion 210 to guide the depressible element while moving between a depressed state and an extended state.

In some examples, the gripping tool may have one or more operational support features configured to aid in the alignment and/or operation of the gripping tool. Examples of operational support features are shown in FIG. 17 as a raised region 225 of the activation element 205 and a raised region of an inferior portion 226 of the gripping tool body portion 210. In this exemplary arrangement of operational support features, the raised regions may be recognized in the orientation of the gripping tool during use. For example, a user may grip the gripping tool and position a finger (e.g., their thumb) on the activation element such that the distal end of their thumb abuts the support feature in a tactile confirmation of the gripping tool orientation relative to the elongate member being controlled and the grip position of the user to the gripping tool. In some examples, the operational support features may increase the capacity for lateral force applicable to the gripping tool in maneuvering an elongate member retained therethrough.

FIG. 18 shows a rear view of the gripping tool 200 of FIG. 17. Operational support features 225 are illustrated in an example arrangement such that the superior operational support feature is distal to the visible side of the gripping tool 200. The body portion 210 can be configured to accept an elongate member (e.g., a catheter) extending into and/or through the gripping tool at an opening 230 in the body portion 210. In some examples, the opening 230 may be an aperture, lumen, passage, channel, tunnel, tube, or other structural aspect of the gripping tool configured to accept, route, or otherwise facilitate the elongate member extending therethrough.

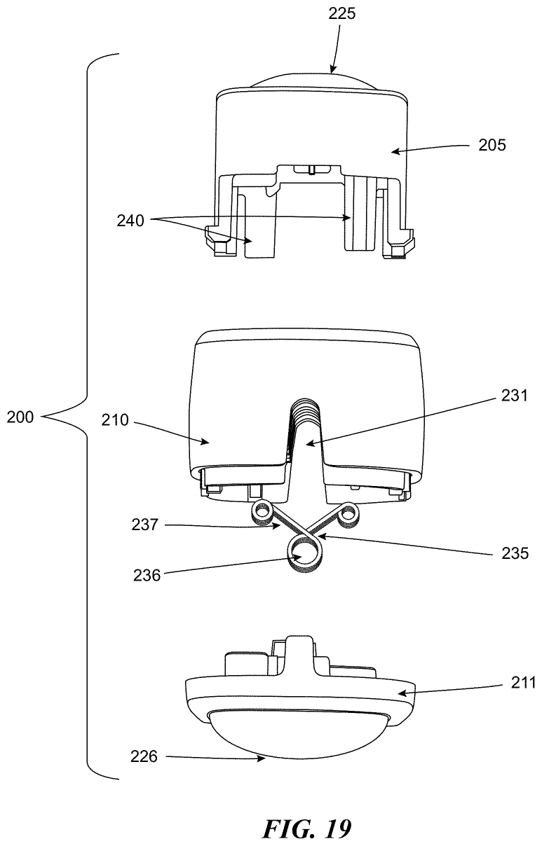

FIG. 19 is an exploded view of the gripping tool 200 of FIGS. 17 and 18 showing some of the internal features. The housing comprises three main portions: the activation element 205, the body portion 210, and a base 211. The base 211 can be coupled to the body portion 210 at a side of the gripping tool 200 generally opposite of the activation element 205. The opening 230 is defined by a generally concave channel extending laterally through body portion 210 and a corresponding protrusion extending upward from base 211 to form an opening or passage for an elongate member such as a catheter to extend therethrough.

In some examples, the base 211 may be configured to couple to the body portion 210 and may provide a mount or foundation for a retention element 235. Alignment elements 240 may be configured to engage corresponding alignment elements of the body portion 210 and/or base 211. The alignment elements may be configured to stabilize operation (e.g., depression) of the activation element 205. For example, as the activation element 205 is depressed against the body portion 210 and base 211, the alignment elements 240 may slidingly engage corresponding alignment elements to promote consistent depression and retraction of the activation element 205.

The retention element may comprise one or more elongate member retention elements. For example, in FIG. 19, an exemplary retention element 235 comprises one or more springs (e.g., torsion springs) forming an adjustable aperture 236 configured to engage an exterior surface of an elongate member (e.g., catheter) passing therethrough. In some examples, when the activation element 205 is depressed to apply a downward force against the spring bias of the torsion spring arms 237, arms 237 move apart and reduce the diameter of aperture 236, thereby applying a radial force against the exterior surface of an elongate member disposed in the aperture 236. In some examples, when activation element 205 is released, the arms 237 move back toward their at-rest position, moving element 205 upward and increasing the diameter of aperture 236, thereby releasing any elongate member extending through aperture 236.

In some examples, the force applied by the retention elements may be measurable in pounds (lb) of force. In some examples, a retention element may be configured to supply a force of 1 lb, 5 lb, 10 lb, 15 lb, 20 lb, 25 lb, 30 lb, 35 lb, 40 lb, or any amount of force in between. In some examples, the suppliable force of a retention elements may be measurable in Newton-meters (Nm) of force. In some examples, a retention element may be configured to supply a torsional force of 0-1 Nm.

In some examples, the amount of force applied to the retention element (e.g., torsion springs) by the activation element is modulated by the user and their grip strength on the gripping tool. In some examples, the retention element is configured to increase a grip force (e.g., radial force) on an elongate member extending therethrough when the activation element is depressed. In some examples, the retention element is configured to decrease a grip force (e.g., radial force) on an elongate member extending therethrough when the activation element is depressed. In some examples, the retention element is configured to decrease a grip force (e.g., radial force) on an elongate member extending therethrough when the activation element is released (e.g., retracted). In some examples, the retention element is configured to increase a grip force (e.g., radial force) on an elongate member extending therethrough when the activation element is released (e.g., retracted).

In some examples, one or more retention elements may comprise a retention assembly. For example, a retention assembly can have one or more retention elements in operable communication with the activation element (e.g., depressible portion) and be configured to engage an elongate member. In some examples, a retention assembly may have one or more spring biased retention elements (e.g., torsion springs) configured to supply a force configured to selectively retain an elongate member. In some examples, a retention assembly may have a retention element (e.g., strap, sling, etc.) and a compression mechanism configured to be selectively adjustable by the activation element to initiate and/or increase a compression force to engage the elongate member. In some examples, the activation element can be configured to reduce and/or eliminate the compression force on the elongate member supplied by the retention element.

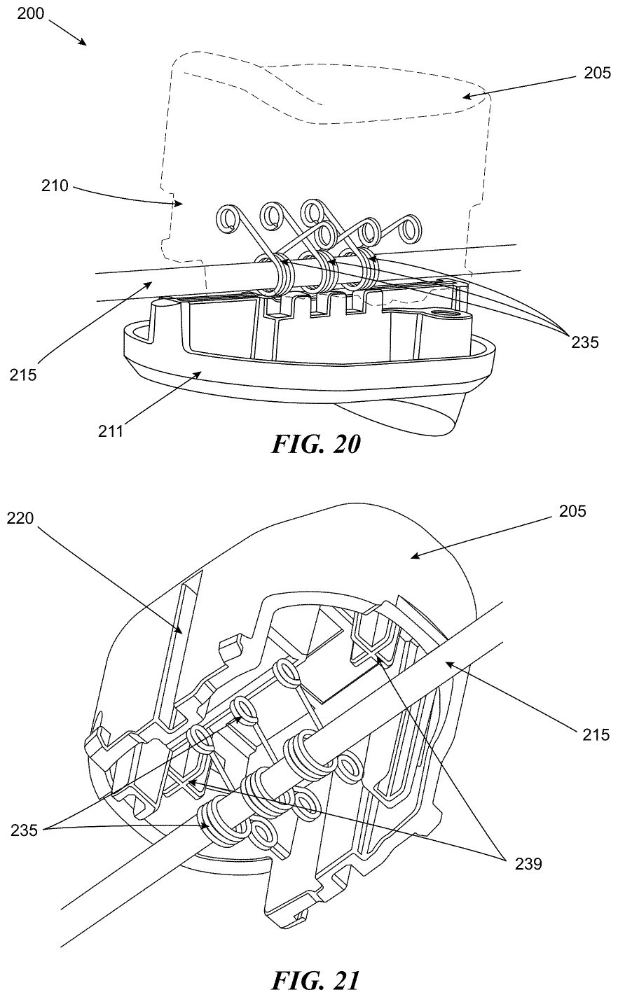

FIG. 20 shows gripping tool 200 illustrated with translucent activation element 205 (for visualization purposes) and body portion 210. In this example, three retention elements are provided in operable communication with an elongate member 215 extending through the retention elements 235. Here, an example of operation can be seen where each retention element 235 is in operable communication with the activation element 205 and the base 211 such that the arms of each torsion spring are contacting the activation element 205 in preparation for selectable operation to adjust each aperture and thereby the radial force (e.g., grip) on the elongate member 215 extending therethrough. Although this example shows three retention elements in the gripping tool, it should be understood that other embodiments can include any number of retention elements, including one, two, three, four, five, six, seven, eight, nine, ten, or more retention elements within the gripping tool.

FIG. 21 is a perspective view of gripping tool 200 with base 211 and body portion 210 removed to illustrate the interaction between the retention elements 235 and the activation element 205. The elongate member 215 can extend through the retention elements 235 as shown. In some examples, the activation element 205 has one or more alignment elements 239 disposed within an interior of the activation element 205 and configured to engage a corresponding alignment element of the body portion, base, or other portion of the gripping tool housing.

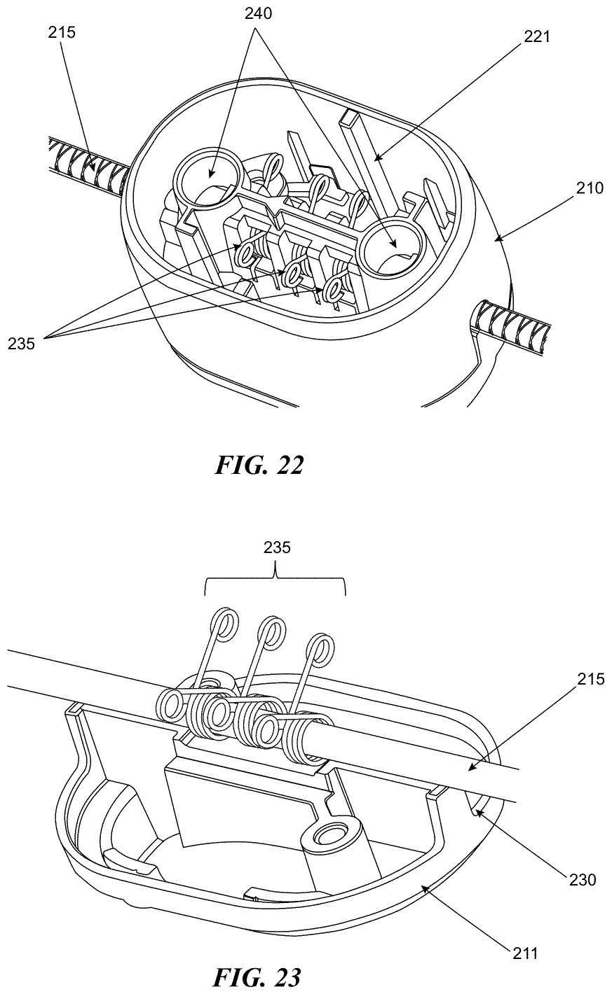

FIG. 22 is a perspective view of gripping tool 200 illustrated without the activation element 205 to expose details in the body portion 210. Corresponding alignment elements 240 in the body portion can be configured to operably communicate with or receive alignment elements of the activation elements 239 of the activation element 205 (shown in FIG. 21). Here, the elongate member 215 is shown extending through the body portion 210 and the retention elements 235. In some examples, additional alignment elements 221 can be configured to communicate with the activation element for consistent and smooth operation (e.g., depression and/or retraction).

FIG. 23 is a perspective view of gripping tool 200 illustrated without activation element 205 or body portion 210. The retention elements 235 forming the retention assembly are supported by base 211. The elongate member 215 (e.g., catheter) extends through the apertures in the retention elements, as shown. In some examples, the base may further comprise a channel, groove, or other structural feature to stabilize the elongate member extending therethrough.

In some examples, the depressible portion of the gripping tool can be configured such that when it is depressed the force supplied by the retention elements is released, reduced, dissipated, or otherwise adjusted to allow the gripping tool to move independent of the sheathing catheter and/or to allow the sheathing catheter to move independent of the gripping tool. For example, the depressible portion can be depressed causing the retention elements to release the sheathing catheter allowing the sheathing catheter to slide/advance through the gripping tool.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The terms “about,” “substantially,” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system for inserting a collapsible blood pump into a patient, the system comprising:
an introducer, the introducer comprising an introducer hub and an introducer sheath extending distally from the introducer hub, the introducer sheath comprising an introducer sheath lumen, the introducer hub comprising a hub connector and a distal hub lumen surrounding a proximal end of the introducer shaft; and
a transfer tool comprising a transfer sheath, the transfer sheath comprising a transfer sheath lumen having a diameter substantially equal to a diameter of the introducer sheath lumen and a transfer tool connector adapted to connect to the hub connector, a distal portion of the transfer sheath extending into the hub when the transfer tool connector is connected to the hub connector.

2. The system of claim 1, wherein the distal portion of the transfer sheath extends into the distal hub lumen when the transfer tool connector is connected to the hub connector.

3. The system of claim 1, wherein the distal end of the transfer sheath abuts a proximal end of the introducer sheath when the transfer tool connector is connected to the hub connector.

4. The system of claim 1, wherein the introducer hub further comprises a tapered surface extending proximally and radially outwardly from the distal hub lumen.

5. The system of claim 1, wherein the introducer further comprises a one-way valve disposed in the introducer hub proximal to the introducer sheath lumen and configured to seal against vascular pressure.

6. The system of claim 1, wherein the introducer further comprises a seal disposed in the introducer hub proximal to the introducer sheath and configured to seal against vascular pressure around a range of diameters of devices inserted through the seal.

7. The system of claim 1, wherein the introducer further comprising a disc valve disposed in the introducer hub proximal to the introducer sheath and configured to seal against vascular pressure around a range of diameters of devices inserted through the valve.

8. The system of claim 1, wherein the introducer hub further comprises a purge fluid port in fluid communication with the distal hub lumen.

9. The system of claim 1, wherein the hub connector comprises threads disposed on the introducer hub.

10. The system of claim 1, wherein the hub connector and transfer tool connector are configured to provide an axial force to move the transfer sheath and introducer sheath toward each other.

11. The system of claim 1, wherein the transfer tool further comprises a proximal hub surrounding a proximal portion of the transfer sheath.

12. The system of claim 11, wherein the transfer tool proximal hub comprises a central lumen, the proximal portion of the transfer sheath being disposed in the central lumen, the central lumen having a reduced diameter portion proximal to a proximal end of the transfer sheath.

13. The system of claim 12, wherein the transfer tool proximal hub further comprises a purge fluid port communicating with the central lumen.

14. The system of claim 12, wherein the transfer tool proximal hub further comprises a seal adapted to seal around a catheter portion of a blood pump.

15. The system of claim 1, wherein the transfer tool further comprises a handle surrounding the transfer sheath.

16. The system of claim 15, wherein the handle extends proximally from the transfer tool connector.

17. The system of claim 16, wherein the transfer tool further comprises a proximal hub, the handle extending from the transfer tool connector to the proximal hub.

18. The system of claim 15, wherein the transfer tool connector comprises threads disposed at a distal end of the handle, the distal portion of the transfer sheath extending distally beyond the transfer tool connector.

19. The system of claim 18, wherein the transfer tool connector comprises a rotatable ring with internal threads.

20. The system of claim 1, wherein the distal portion of the transfer sheath is radially expandable.

21. A method of deploying an expandable blood pump in a patient, the blood pump comprising an expandable and compressible pump housing, an impeller disposed in the pump housing, and a catheter extending proximally from the pump housing, the method comprising:
moving at least a portion of the pump housing proximally into a transfer sheath of a transfer tool through a distal opening of the transfer sheath, the pump housing compressing as it enters the transfer sheath;
advancing the transfer sheath distally into a hub of an introducer sheath disposed in a blood vessel of the patient;
advancing the pump housing out of the transfer sheath into the introducer sheath; and
advancing the pump housing out of the introducer sheath and into the blood vessel.

22. The method of claim 21 wherein the transfer sheath advances distally into the hub of the introducer until a distal end of the transfer sheath abuts a proximal end of the introducer sheath.

23. The method of claim 21, wherein the transfer sheath comprises a transfer sheath lumen and the introducer sheath comprises an introducer sheath lumen, the transfer sheath lumen having a diameter substantially equal to a diameter of the introducer sheath lumen.

24. The method of claim 21, further comprising connecting a connector of the transfer tool to a connector of the introducer hub.

25. The method of claim 24, wherein the connecting step comprises applying an axial force to move the transfer sheath and introducer sheath toward each other.

26. The method of claim 21, further comprising expanding the distal end of the transfer sheath as the pump housing moves into the transfer sheath.

27. The method of claim 21, further comprising compressing the distal end of the transfer sheath before the distal end of the transfer sheath abuts the proximal end of the introducer sheath.

28. The method of claim 21, wherein the step of moving the pump housing proximally into the transfer sheath further comprises moving the pump housing proximally until proximal struts of the pump housing engage a sheathing stop at a proximal end of the transfer sheath.

29. The method of claim 21, further comprising injecting purge fluid into a proximal end of the transfer sheath while the pump housing is disposed in the transfer sheath.

30. A method of deploying an expandable blood pump in a patient, the blood pump comprising an expandable and compressible pump housing, an impeller disposed in the pump housing, and a catheter extending proximally from the pump housing, the method comprising:
moving the pump housing proximally into a transfer sheath of a transfer tool through a distal opening of the transfer sheath, the pump housing compressing as it enters the transfer sheath and a distal end of the transfer sheath expanding as the pump housing enters the transfer sheath;
advancing the transfer sheath distally into a hub of an introducer sheath disposed in a blood vessel of the patient;
compressing the distal end of the transfer sheath within the hub;
advancing the pump housing out of the transfer sheath into the introducer sheath; and
advancing the pump housing out of the introducer sheath and into the blood vessel.

31. The method of claim 30, wherein the transfer sheath comprises a transfer sheath lumen and the introducer sheath comprises an introducer sheath lumen, the transfer sheath lumen having a diameter substantially equal to a diameter of the introducer sheath lumen after the compressing step.

32. The method of claim 30, further comprising connecting a connector of the transfer tool to a connector of the introducer hub.

33. The method of claim 32, wherein the connecting step comprises applying an axial force to move the transfer sheath and introducer sheath toward each other.

34. The method of claim 30, further comprising compressing the distal end of the transfer sheath before the distal end of the transfer sheath abuts the proximal end of the introducer sheath.

35. The method of claim 30, wherein the step of moving the pump housing proximally into the transfer sheath further comprises moving the pump housing proximally until proximal struts of the pump housing engage a sheathing stop at a proximal end of the transfer sheath.

36. The method of claim 30, further comprising injecting purge fluid into a proximal end of the transfer sheath while the pump housing is disposed in the transfer sheath.
